(19) United States
(12) Patent Application Publication
(10) Pub. No.: US 2013/0040912 A1
(43) Pub. Date: Feb. 14, 2013

(54) PIPERAZINOTRIAZINES AS PI3K INHIBITORS FOR USE IN THE TREATMENT ANTIPROLIFERATIVE DISORDERS

(71) Applicants: Vladimir Cmiljanovic, Basel (CH); Natasa Cmiljanovic, Basel (CH); Bernd Giese, Fribourg (CH); Matthias Wymann, Bern (CH)

(72) Inventors: Vladimir Cmiljanovic, Basel (CH); Natasa Cmiljanovic, Basel (CH); Bernd Giese, Fribourg (CH); Matthias Wymann, Bern (CH)

(73) Assignee: UNIVERSITY OF BASEL, Basel (CH)

(21) Appl. No.: 13/643,357

(22) PCT Filed: Apr. 27, 2011
(86) PCT No.: PCT/IB2011/051829

§ 371 Date: Oct. 25, 2012

(30) Foreign Application Priority Data
Apr. 30, 2010 (GB) 1007227.0

(51) Int. Cl. C07D 413/14; A61K 31/5377; A61K 31/69; A61P 35/00; A61P 3/10; G01N 33/573; A61P 25/28; A61P 9/10; A61P 17/06; A61P 37/08; A61P 35/02; A61P 1/16; C07F 5/02; A61P 9/00

(52) U.S. Cl. 514/64; 544/113; 544/69; 514/234.5; 435/7.8

(57) ABSTRACT

The invention relates to compounds of formula (I) R1 is methyl, n-hexyl, aminoethyl, methylaminoethyl, ethylaminoethyl, dimethylaminoethyl, acryloylaminoethyl, methacryloylaminoethyl, methoxyethyl, ethoxyethyl, d-C4-alkyl-sulfonyl, acryloyl, or methacryloyl; or R1 is aminoethyl, acryloyl or acryloylaminoethyl carrying a linker and a tag, and R2 and R3, independently of each other, are hydrogen or CrC4-alkyl, or R2 and R3 together form a methylene or an ethylene bridge; and tautomers, solvates and pharmaceutically acceptable salts thereof. These compounds are effective in preventing or treating a disease or disorder modulated by PI3 kinases and/or mTOR, in particular treating a hyperproliferative disorder.

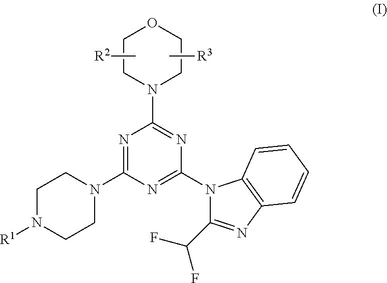

### FIELD OF THE INVENTION

The invention relates to new triazines carrying a benzimidazo, a morpholino and a 4-substituted piperazino substituent, which inhibit phosphoinositide 3-kinase (PI3K), mammalian target of rapamycin (mTOR), DNA-PK and ATM kinase, and to pharmaceutically acceptable salts thereof. The invention also relates to methods of using the compounds for the treatment of associated pathological conditions.

### BACKGROUND OF THE INVENTION

Protein kinases participate in the signaling events which control the activation, growth, differentiation, survival and migration of cells in response to extracellular mediators or stimuli including growth factors, cytokines or chemokines. In general, these kinases are classified in two groups, those that preferentially phosphorylate tyrosine residues and those that preferentially phosphorylate serine and/or threonine residues. The tyrosine kinases include membrane-spanning growth factor receptors, for example the epidermal growth factor receptor (EGFR) and cytosolic non-receptor kinases including Src family kinases, the Syk family kinases and the Tec family kinases.

Inappropriately high protein kinase activity is involved in many diseases including cancer, metabolic diseases, immunological diseases and inflammatory disorders. This can be caused either directly or indirectly by the failure of control mechanisms due to mutation, overexpression or inappropriate activation of the enzyme.

Phosphoinositide 3-kinases (PI3Ks) have been recognized to modulate a wide range of cellular activities, and to be central to the growth and metabolic control. Genetically modified mice targeting the PI3K pathway, and the elucidation of human hereditary disease like Cowden's syndrome, tuberous sclerosis, ataxia telangiectasia, X-linked myotubular myopathy and Charcot-Marie-Tooth neuropathy, have provided further insight into the cellular and systemic role of phosphoinositide signaling. Deregulation of phosphoinositide levels, and in particular the product of class I PI3Ks, PtdIns (3,4,5)P3, is involved in the pathogenesis of cancer, chronic inflammation, allergy, metabolic disease, diabetes and cardiovascular problems.

The PI3 kinase/Akt/PTEN pathway is an attractive target for cancer drug development since such agents would be expected to inhibit proliferation, reverse the repression of apoptosis and surmount resistance to cytotoxic agents in cancer cells. PI3 kinase inhibitors have been reported [see notably Marone et al., Biochimica et Biophysica Acta 1784:159-185 (2008)].

Certain pyrimidine compounds (WO 2008/032033) and triazine compounds (WO 02/088112, WO 2004/03782, WO 2006/095906) are known to have PI3K and/or mTOR inhibitor activity and inhibit the growth of cancer cells. The triazine compound ZSTK474 (Zenyaku Kogyo) is the first orally administered triazine compound highly active against PI3Ks that displayed potent antitumor activity against human cancer xenografts in mice, without evidence of critical toxicity [Yaguchi et al., Journal of the National Cancer Institute, 98:545-556, (2006)]. ZSTK474 is an ATP-competitive inhibitor of class I phosphatidyl-inositol 3-kinase isoforms [Kong et al., Cancer Sci, 98:1638-1642 (2007)].

### SUMMARY OF THE INVENTION

The invention relates to compounds of formula (I)

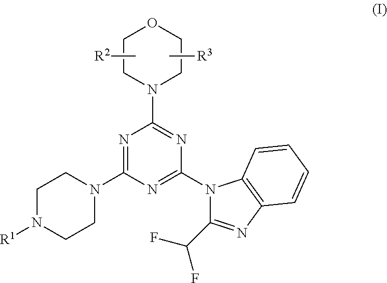

wherein
R¹ is methyl, n-hexyl, aminoethyl, methylaminoethyl, ethylaminoethyl, dimethylaminoethyl, acryloylaminoethyl, methacryloylaminoethyl, methoxyethyl, ethoxyethyl, C₁-C₄-alkyl-sulfonyl, acryloyl, or methacryloyl; or R¹ is aminoethyl, acryloyl or acryloylaminoethyl carrying a linker and a tag, and
R² and R³, independently of each other, are hydrogen or C₁-C₄-alkyl, or R² and R³ together form a methylene or an ethylene bridge;
and tautomers, solvates and pharmaceutically acceptable salts thereof.

Further the invention relates to pharmaceutical compositions comprising a compound of formula (I), and methods of preventing or treating a disease or disorder modulated by PI3 kinases and/or mTOR, in particular treating a hyperproliferative disorder, comprising administering to a mammal in need of such treatment an effective amount of a compound of formula (I). An additional aspect of the invention is the use of a compound of formula (I) for the treatment or prevention of a disease or condition modulated by PI3 kinase and/or mTOR in a mammal, and the use of a compound of formula (I) in the preparation of a medicament for the treatment or prevention of a disease or condition modulated by PI3 kinase and/or mTOR in a mammal.

Another aspect of the invention includes methods of preparing, methods of separating, and methods of purifying compounds of formula (I), novel intermediates useful for preparing compounds of formula (I) as defined hereinbefore, and methods of screening for screening for lipid kinase inhibitors.

### DETAILED DESCRIPTION OF THE INVENTION

The term “C₁-C₄-alkyl” as used herein refers to a saturated linear or branched-chain monovalent hydrocarbon radical of one to four carbon atoms. Examples of C₁-C₄-alkyl are methyl, ethyl, n-propyl, iso-propyl, n-butyl, iso-butyl, sec-butyl, and tert-butyl.

The term “C₁-C₄-alkylsulfonyl” means C₁-C₄-alkyl as defined above connected to an —SO₂— group, which is attached to the position 4 of the piperazine shown in formula (I).

In aminoethyl, methylaminoethyl, ethylaminoethyl, dimethylaminoethyl, acryloylaminoethyl, methacryloylaminoethyl, methoxyethyl, ethoxyethyl, and acryloylaminoethyl, the amino or alkoxy function is preferably connected to the 2-position of ethyl.

The R² and R³ substituents may be connected to any carbon atom of the morpholine shown in formula (I), as indicated by the bond pointing to the center of morpholine and not connected to a particular position. Examples of such carbon atom are those in positions 2, 3, 5 and 6. The R² and R³ substituents may be located on different carbon atoms, or on the same carbon atoms giving rise to geminal substitution. Examples for positions for substituents R²/R³ are 2/2, 2/3, 2/5, 2/6, 3/3, and 3/5. If R² and R³ together represent ethylene, the same positions are possible, whereby geminal substitution leads to a spiro cyclopropyl function, vicinal substitution leads to an annulated cyclobutane, or the preferred 2/5, 2/6 and 3/5 substitution leads to truly bridged compounds. If R² and R³ together represent methylene, positions 2/3, 2/5, 2/6, and 3/5 are possible, whereby vicinal substitution leads to an annulated cyclopropane, or the preferred 2/5, 2/6 and 3/5 substitution leads to truly bridged compounds.

The term “chiral” refers to molecules, which have the property of non-identity of the mirror image, while the term “achiral” refers to molecules, which are superimposable on their mirror image.

The term “stereoisomers” refers to compounds, which have identical chemical constitution, but differ with regard to the arrangement of the atoms or groups in space. “Diastereomer” refers to a stereoisomer with two or more centers of chirality. Diastereomers are not mirror images of one another, and they have different physical properties, e.g. melting points, boiling points, spectral properties, and reactivities. Mixtures of diastereomers may be separated by crystallization or with high resolution analytical procedures such as electrophoresis and chromatography. “Enantiomers” refer to two stereoisomers of a compound which are non-superimposable mirror images of one another.

Stereochemical definitions and conventions used herein generally follow S. P. Parker, Ed., McRaw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and I or (+) and (−) are employed to designate the sign of rotation of plane-polarized light by the compound, with (−) or I meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate.

The term “tautomer” or “tautomeric form” refers to structural isomers of different energies, which are interconvertible via a low energy barrier. For example, proton tautomers include interconversions via migration of a proton, such as keto-enol and imin-enamine isomerizations.

The term “pharmaceutically acceptable salt” as used herein, refers to pharmaceutically acceptable organic or inorganic salts of a compound of the invention. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an α-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

The term “pharmaceutically acceptable” indicates that the substance or composition must be compatible chemically and/or toxicologically, with the other ingredients comprising a formulation, and/or the mammal being treated therewith.

A “solvate” refers to an association or complex of one or more solvent molecules with a compound of the invention. Examples of solvents that form solvates include, but are not limited to, water, isopropanol, ethanol, methanol, DMSO, ethyl acetate, acetic acid, and ethanolamine. The term “hydrate” refers to the complex wherein the solvent molecule is water.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl, and 9-fluorenylmethylenoxycarbonyl (Fmoc). For a general description of protecting groups and their use, see T. W. Greene, Protective Groups in Organic Synthesis, John Wiley & Sons, New York, 1991.

The term “linker” includes, but is not limited to, a chain of 1 to 20, preferably 2 to 6, optionally substituted methylene groups, or such chain wherein one or more methylene groups are replaced by oxygen, a carbonyloxy group, optionally substituted nitrogen, a carboxamide group, a urea group, sulphur, a disulfide group, or combinations thereof. Substituents considered are oxo (giving a carbonyl function), C₁-C₆ alkyl, a chain of 1 to 6 methylene groups giving rise to a trifunctional linker, phenyl, phenylene giving rise to a trifunctional linker, or residues of naturally occurring amino acids. Particular linkers are, e.g., a polymethylene group, a polymethylene group comprising one or two amide functions, a polyoxyethylene group, or a small peptide consisting of one to six of the naturally occurring 20 essential amino acids. The linker is connected to the beta-carbon atom of the acryloyl group or to the amino group in aminoethyl. “A linker carrying a tag” means a linker connected to the beta-carbon atom of acryloyl or to the amino group in aminoethyl at one end and a tag at the other end of the linker, or being a trifunctional linker carrying two different tags.

The term “tag” includes, but is no limited to biotin, avidin, streptavidin, a fluorescent marker, or a solid phase, for example a polymeric bead or a plastic or glass slide. Examples of fluorescent markers considered are 4,4-difluoro-1,3,5,7-tetramethyl-4-bora-3a,4a-diaza-s-indacene-8-propionic acid (BODIPY® 493/503, SE), 4,4-difluoro-5,7-dimethyl-4-bora-3a,4a-diaza-s-indacene-3-propionic acid (BODIPY® FL), 4,4-difluoro-5,7-dimethyl-4-bora-3a,4a-diaza-s-indacene-3-propionic acid (BODIPY® FL, SE), 6-((4,4-difluoro-5,7-dimethyl-4-bora-3a,4a-diaza-s-indacene-3-propionyl)amino)hexanoic acid (BODIPY® FL-X, SE), 4,4-difluoro-5-phenyl-4-bora-3a,4a-diaza-s-indacene-3-propionic acid (BODIPY® R6G, SE), 4,4-difluoro-5,7-diphenyl-4-bora-3a,4a-diaza-s-indacene-3-propionic acid (BODIPY® 530/550, SE), 6-((4,4-difluoro-1,3-dimethyl-5-(4-methoxyphenyl)-4-bora-3a,4a-diaza-s-indacene-2-propionyl)amino)hexanoic acid (BODIPY® TMR-X, SE), 4,4-difluoro-5-(2-thienyl)-4-bora-3a,4a-diaza-s-indacene-3-propionic acid (BODIPY® 558/568, SE), 4,4-difluoro-5-styryl-4-bora-3a,4a-diaza-s-indacene-3-propionic acid (BODIPY® 564/570, SE), 6-(((4-(4,4-difluoro-5-(2-thienyl)-4-bora-3a,4a-diaza-s-indacene-3-yl)phenoxy)acetyl)amino)hexanoic acid (BODIPY® TR-X, SE), 6-(((4,4-difluoro-5-(2-thienyl)-4-bora-3a,4a-diaza-s-indacene-3-yl)styryloxy)acetyl)aminohexanoic acid (BODIPY® 630/650-X, SE), Alexa Fluor® 350 carboxylic acid, 5-carboxyrhodamine 6G (5-CR 6G, SE), Rhodamine Green™ carboxylic acid, hydrochloride (5(6)-CR 110, SE), which are usually applied as succinimidyl esters for reaction with a linker containing an amine functional group at one end.

The term “treat” and “treatment” refer to both therapeutic treatment and prophylactic or preventative measures, wherein the object is to prevent or slow down (lessen) an undesired pathological change or disorder, such as the development or spread of cancer. For purpose of this invention, beneficial or desired clinical results include, but are not limited to, alleviation of symptoms, diminishment of extent of disease, stabilizing (i.e., not worsening) the disease state, delay or slowing of disease progression, amelioration or palliation of the disease state, and partial or total remission, whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukaemia or lymphoid malignancies. More particular examples of such cancers include squamous cell cancer (e.g., epithelial squamous cell cancer), lung cancer including small-cell lung cancer, non-small-cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatome, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of known chemotherapeutic agents include trastuzumab, pertuzumab, erlotinib, bortezomib, fulvestrant, sunitib, letrozole, imatinib mesylate, finasunate, oxaliplatin, 5-fluorouracil, leucovorin, rapamycin, lapatinib, lonafarnib, sorafenib, gefitinib, AG1478, alkylating agents such as thiotepa, cyclophosphamide; alkyl sulfonates such as busulfan, improsulfan and piposulfan; aziridines such as benzodopa, carboquone, meturedopa, and uredopa; ethyleneimines and melamines including altretamine, triethylenemelamine, triethylenephosphoramide, triethylenethiophosphoramide and trimethylomelamine; acetogenins; a camptothecin (including the synthetic analog topotecan); bryostatin; callystatin; CC-1065 (including the synthetic analogs adozelesin, carzelesin and bizelesin); cryptophycins; dolastatin; duocarmycin (including the synthetic analogs KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammal and calicheamicin omegal; dynemicin, including dynemicin A; biphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophillin, chromomycinis, dactinomycin, daunorubicin, detorubicin, 6-diazol-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil; folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofuran; spirogermanium; tenuazonic acid; triaziquone; trichothecenes; urethane; vindesine; dacarbazine; mannomustine; mitobronitol; mitolactol; pipobroman; gacytosine; arabinoside; taxoids, e.g., paclitaxel, albumin-engineered nanoparticle formulations of paclitaxel, and docetaxel, doxetaxel; chlorambucil; gemcitabine; 6-thioguanine; mercaptopurine; methotrexate; platinum analogs such as cisplatin and carboplatin; vinblastine; etoposide; ifosfamide; mitoxantrone; vincristine; vinorelbine; novantrone; teniposide; edatrexate; daunomycin; aminopterin; capecitabine; ibandronate; CP-11; topoisomerase inhibitor RFS 2000; difluoromethylornithine (DMFO); retinoids such as retinoic acid; and pharmaceutically acceptable salts; acids and derivatives of any of the above.

Also included in the definition of “chemotherapeutic agent” are: (i) anti-hormonal agents that act to regulate or inhibit hormone action on tumors such as anti-estrogens and selective receptor modulators (SERMs), including, for example, tamoxifen, tamoxifen citrate, raloxifene, droloxifene, and toremifine citrate; (ii) aromatase inhibitors that inhibit the enzyme aromatase, which regulates estrogen production in the adrenal glands, such as, for example, 4(5)-imidazoles, megestrol acetate; exemestane; formestanie, fadrazole, vorozole, letrozole, and anastrozole; (iii) anti-androgens such as flutamide, nilutamide; (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Raf1 and H-Ras; (vii) ribozymes such as VEGF expression inhibitors and HER2 expression inhibitors; (viii) vaccines such as gene therapy vaccines, for example, plasmid/lipid complex containing the DNA sequences encoding HLA-B7 and β2 microglobulin, or DNA sequences encoding interleukin-2, aldesleukin (rIL-2); a topoisomerase 1 inhibitor such as lurtotecane or abarelix; (ix) anti-angiogenic agents such as bevacizumab; and (x) pharmaceutically acceptable salts, acids and derivatives of any of the above.

A “liposome” is a small vesicle composed of various types of lipids, phospholipids and/or surfactant, which is useful for delivery of a drug (such as the PI3K and mTOR kinase inhibitors disclosed herein and, optionally, a chemotherapeutic agent) to a mammal. The components of the liposome are commonly arranged in a bilayer formation, similar to the lipid arrangement of biological membranes.

The term “package insert” is used to refer to instructions customarily included in commercial packages of therapeutic products, that contain information about the indications, usage, dosage, administration, contraindications and/or warnings concerning the use of such therapeutic products.

The term “mammal” includes, but is not limited to, humans, mice, rats, guinea, pigs, monkeys, dogs, cats, horses, cows, pigs, and sheep.

The invention relates to compounds of formula (I)

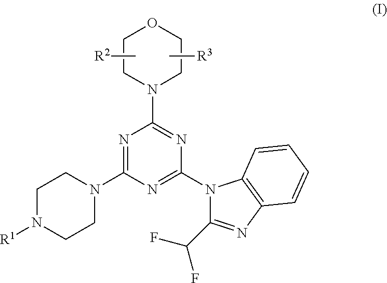

wherein
R¹ is methyl, n-hexyl, aminoethyl, methylaminoethyl, ethylaminoethyl, dimethylaminoethyl, acryloylaminoethyl, methacryloylaminoethyl, methoxyethyl, ethoxyethyl, C₁-C₄-alkylsulfonyl, acryloyl, or methacryloyl; or R¹ is aminoethyl, acryloyl or acryloylaminoethyl carrying a linker and a tag, and
R² and R³, independently of each other, are hydrogen or C₁-C₄-alkyl, or R² and R³ together form a methylene or an ethylene bridge;
and tautomers, solvates and pharmaceutically acceptable salts thereof.

Preferred are compounds wherein R¹ is methyl, n-hexyl, 2-aminoethyl, 2-(methylamino)ethyl, 2-(ethylamino)ethyl, 2-(acryloylamino)ethyl, 2-methoxyethyl, 2-ethoxyethyl, C₁-C₄-alkylsulfonyl, acryloyl, or methacryloyl; or R¹ is 2-aminoethyl, acryloyl or 2-(acryloylamino)ethyl, each carrying a linker and a tag.

More preferred are compounds wherein R¹ is methyl, 2-aminoethyl, 2-(acryloylamino)ethyl, 2-ethoxyethyl, methylsulfonyl, ethylsulfonyl, iso-propylsulfonyl, acryloyl, or methacryloyl, in particular methyl, 2-aminoethyl, 2-(acryloylamino)ethyl, 2-ethoxyethyl, methylsulfonyl, ethylsulfonyl, or acryloyl, more particularly methyl, 2-(acryloylamino)ethyl, methylsulfonyl, or acryloyl.

Also preferred are compounds wherein R¹ is 2-aminoethyl or acryloyl, each carrying a linker and a tag, in particular wherein the tag is biotin, a fluorophore or a polymeric bead.

Further preferred are compounds wherein R² and R³, independently of each other, are hydrogen, methyl, ethyl, or isopropyl, or R² and R³ together form a methylene or an ethylene bridge. In particular, R² and R³ are both hydrogen, one hydrogen and one methyl, or both methyl, or form together an ethylene bridge.

More preferably R² is (S)-2-methyl, (R)-2-methyl, (R)-3-methyl, or (S)-3-methyl, and R³ is hydrogen, or R² and R³ are (2R,6S)-2,6-dimethyl, (2R,6R)-2,6-dimethyl, (2R,3R)-2,3-dimethyl, (2S,5S)-2,5-dimethyl, (3S,5R)-3,5-dimethyl, (3S,5S)-3,5-dimethyl, a 2,5-ethylene bridge, a 2,6-ethylene bridge, a 3,5-ethylene bridge, or both hydrogen. Most preferably, both R² and R³ are hydrogen.

Most preferred are compounds of formula (I) wherein R¹ is methyl, n-hexyl, 2-aminoethyl, 2-(acryloylamino)ethyl, 2-ethoxyethyl, methylsulfonyl, ethylsulfonyl, or acryloyl, and R² and R³ are hydrogen, in particular the compounds of formula (I) wherein R¹ is methyl, 2-(acryloylamino)ethyl, methylsulfonyl, or acryloyl, and R² and R³ are hydrogen, such as the compounds wherein R¹ is methyl or methylsulfonyl, and R² and R³ are hydrogen.

Equally preferred are compounds of formula (I) wherein R¹ is 2-aminoethyl carrying a linker and biotin, a fluorophore or a polymeric bead, and R² and R³ are hydrogen.

Most preferred are the compounds of the examples.

In the structures shown herein, where the stereochemistry of any particular chiral atom is not specified, then all stereoisomers are contemplated and included as the compounds of the invention.

The compounds of the present invention may exist in unsolvated as well as solvated forms with pharmaceutically acceptable solvents such as water, ethanol, and the like, and it is intended that the invention embraces both solvated and unsolvated forms. The compounds of the invention may also exist in different tautomeric forms (tautomers), and all such forms are embraced with the scope of the invention.

### Methods of Synthesis

The compounds of the invention may be synthesized by synthetic routes that include processes analogous to those well known in the chemical arts, particularly in light of the description contained herein. The starting materials are generally available from commercial sources or are readily prepared using methods well known to those skilled in the art. For illustrative purposes, Scheme 1 shows a general method for preparing the compounds of the present invention as well as key intermediates. The substituents are introduced into the triazine nucleus by sequential replacement of a halogen (Hal), e.g. chlorine or bromine, by the corresponding secondary amine. The sequence of replacement reaction by such a secondary amine is, in principle, freely exchangeable, and may proceed through any pair of intermediates shown in Scheme 1, originally starting with 2,4,6-trihalotriazine (cyanuric chloride or cyanuric bromide). Furthermore, substituent R¹ may be modified at any stage of the reaction sequence.

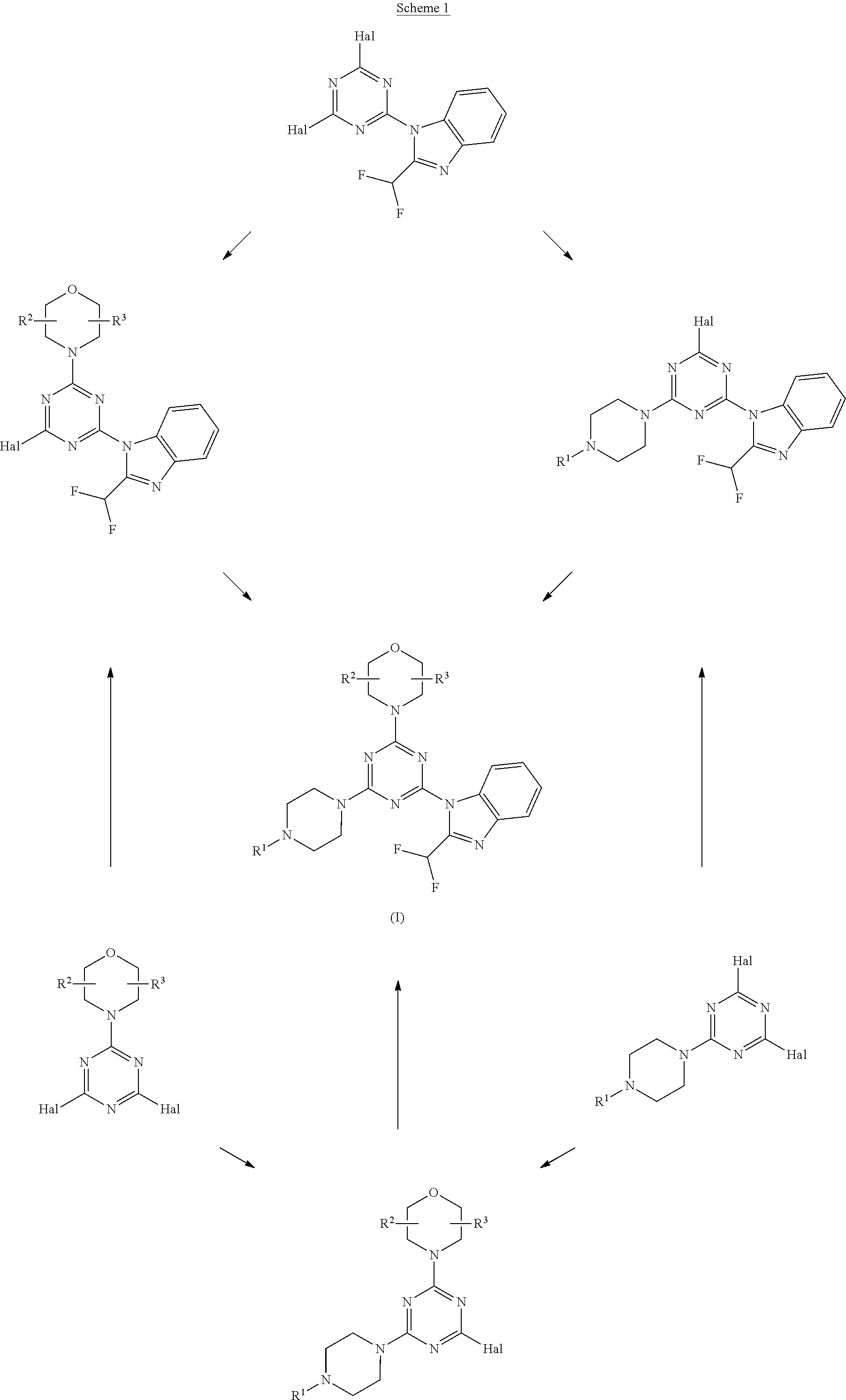

For example, for introduction of the piperazine substituent into the triazine nucleus, one nitrogen atom of piperazine may carry an amino protecting group, which is then subsequently split off giving a compound wherein R¹ means hydrogen, and then further modified by reaction with diazomethane or a methyl halide to convert R¹ to methyl, by reaction with n-hexyl halide to convert R¹ to n-hexyl, by reaction with protected and/or substituted 2-aminoethyl halide to convert R¹ to optionally substituted 2-aminoethyl, by reaction with 2-ethoxy- or 2-methoxyethyl halide to convert R¹ to 2-ethoxy- or 2-methoxyethyl, by reaction with C₁-C₄-alkylsulfonyl halide to convert R¹ to C₁-C₄-alkylsulfonyl, or by reaction with optionally substituted acryloyl halide or anhydride to convert R¹ to acryloyl, methacryloyl or acryloyl carrying a linker with an optionally protected amino function at one end or a tag. R¹ with the meaning 2-aminoethyl may be further elaborated to the desired derivative by reaction with diazomethane, methyl or ethyl halide, acryloyl halide or acryloyl anhydride or a linker carrying halide at one end and a tag or a protected amino function at the other end. An amino function of the linker may, in the last step of the sequence, react with an N-hydroxysuccinimide ester of a tag to give the desired linker carrying a tag. Alternatively, the piperazine used for reaction with a halo-substituted triazine may already carry the final substituent R¹.

For a more detailed description of the individual reaction steps, see the examples hereinbelow. Those skilled in the art will appreciate that other synthetic routes may be used to synthesize the compounds of the invention. Although specific starting materials and reagents are depicted in the scheme and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the methods of preparing the compounds of this invention, it may be advantageous to separate reaction products from one another and/or from starting materials. The desired products of each step or series of steps are separated and/or purified to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography. Another class of separation methods involves treatment of a mixture with a reagent selected from activated carbon, molecular sieves, ion exchange media, or the like.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Also, some of the compounds of the present invention may be atropisomers and are considered as part of this invention. Enantiomers can also be separated by use of a chiral HPLC column.

### Methods of Treatment

The compounds of the invention may be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier or excipient. Where the compound is administered parenterally, it may be formulated with a pharmaceutically acceptable parenteral vehicle and in a unit dosage injectable form, as detailed below.

A dose to treat human patients may range from about 10 mg to about 1000 mg of the compound of the invention. A typical dose may be about 100 mg to about 300 mg of the compound. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

Compounds of the present invention are useful for treating diseases, conditions and/or disorders including, but not limited to, those characterized by over expression of lipid kinases, e.g. PI3 kinase. Accordingly, another aspect of this invention includes methods of treating or preventing diseases or conditions that can be treated or prevented by inhibiting lipid kinases, including PI3K and mTOR. In one embodiment, the method comprises administering to a mammal in need thereof a therapeutically effective amount of a compound of the invention or of pharmaceutical composition comprising it.

Diseases and conditions treatable according to the methods of this invention include, but are not limited to, cancer, stroke, diabetes, hepatomegaly, cardiovascular disease, Alzheimer's disease, cystic fibrosis, autoimmune diseases, atherosclerosis, restenosis, psoriasis, allergic disorders, inflammation, neurological disorders, a hormone-related disease, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), liver disease, pathologic immune conditions involving T cell activation, and CNS disorders in a patient.

Cancers which can be treated according to the methods of this invention include, but are not limited to, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. Cardiovascular diseases which can be treated according to the methods of this invention include, but are not limited to, restenosis, cardiomegaly, atherosclerosis, myocardial infarction, and congestive heart failure. Neurodegenerative disease which can be treated according to the methods of this invention include, but are not limited to, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia. Inflammatory diseases which can be treated according to the methods of this invention include, but are not limited to, rheumatoid arthritis, psoriasis, contact dermatitis, and delayed hypersensitivity reactions.

Another aspect of this invention provides a compound of this invention for use in the treatment of the diseases or conditions described herein in a mammal, for example, a human, suffering from such disease or condition. Also provided is the use of a compound of this invention in the preparation of a medicament for the treatment of the diseases and conditions described herein in a warm-blooded animal, such as a mammal, for example a human, suffering from such disorder.

### Pharmaceutical Compositions

In order to use a compound of this invention for the therapeutic treatment (including prophylactic treatment) of mammals including humans, it is normally formulated in accordance with standard pharmaceutical practice as a pharmaceutical composition. According to this aspect of the invention there is provided a pharmaceutical composition comprising a compound of this invention in association with a pharmaceutically acceptable diluent or carrier.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are nontoxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives.

The formulations may be prepared using conventional dissolution and mixing procedures. For example, the bulk drug substance is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, a compound of the invention having the desired degree of purity may optionally be mixed with pharmaceutically acceptable diluents, carriers, excipients or stabilizers, in the form of a lyophilized formulation, milled powder, or an aqueous solution, formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compound of this invention for use herein is preferably sterile. In particular, formulations to be used for in vivo administration must be sterile. Such sterilization is readily accomplished by filtration through sterile filtration membranes. The compound ordinarily can be stored as a solid composition, a lyophilized formulation or as an aqueous solution. The pharmaceutical compositions of the invention will be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding. As a general proposition, the initial pharmaceutically effective amount of the inhibitor administered parenterally per dose will be in the range of about 0.01-100 mg/kg, namely about 0.1 to 20 mg/kg of patient body weight per day, with the typical initial range of compound used being 0.3 to 15 mg/kg/day.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatine microcapsules and poly-(methylmethacylate) microcapsules, respectively, in colloidal drug delivery systems (for example, liposomes, albumin microspheres, microemulsions, nanoparticles and nanocapsules) or in macroemulsions.

Sustained-release preparations of compounds of the invention may be prepared. Suitable examples of sustained-release preparations include semipermeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or polyvinyl alcohol)), polylactides, copolymers of L-glutamic acid and gamma-ethyl-L-glutamate, non-degradable ethylene-vinyl acetate, degradable lactic acid-glycolic acid copolymers and poly-D-(−)-3-hydroxybutyric acid.

Formulations of a compound of the invention suitable for oral administration may be prepared as discrete units such as pills, capsules, cachets or tablets each containing a predetermined amount of a compound of the invention. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom. Tablets, troches, lozenges, aqueous or oil suspensions, dispersible powders or granules, emulsions, hard or soft capsules, e.g., gelatin capsules, syrups or elixirs may be prepared for oral use. Formulations of compounds of the invention intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include a polyhydric alcohol, i.e., an alcohol having two or more hydroxy groups such as propylene glycol, butane-1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs. The oily phase of the emulsions of this invention may be constituted from known ingredients in a known manner. While the phase may comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of compounds of the invention contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. The aqueous and nonaqueous sterile injection solutions may contain anti-oxidants, buffers, bacteriostats and solutes which render the formulation isotonic with the blood of the intended recipient. Aqueous and non-aqueous sterile suspensions may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below. Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily subdose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Combination Therapy

The compounds of the invention may be employed alone or in combination with other therapeutic agents for the treatment of a disease or disorder described herein, such as a hyperproliferative disorder (e.g., cancer). In certain embodiments, a compound of the invention combined in a pharmaceutical combination formulation, or dosing regimen as combination therapy, with a second compound that has anti-hyperproliferative properties or that is useful for treating a hyperproliferative disorder (e.g., cancer). The second compound of the pharmaceutical combination formulation or dosing regimen preferably has complementary activities to the compound of the invention such that they do not adversely affect each other. Such compounds are suitably present in combination in amounts that are effective for the purpose intended. In one embodiment, a composition of this invention comprises a compound of the invention in combination with a chemotherapeutic agent such as described herein.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities. Suitable dosages for any of the above coadministered agents are those presently used and may be lowered due to the combined action (synergy) of the newly identified agent and other chemotherapeutic agents or treatments.

In a particular embodiment of anti-cancer therapy, a compound of the invention may be combined with other chemotherapeutic, hormonal or antibody agents such as those described herein, as well as combined with surgical therapy and radiotherapy. Combination therapies according to the present invention thus comprise the administration of at least one compound of the invention and the use of at least one other cancer treatment method. The amounts of the compound(s) of the invention and the other pharmaceutically active chemotherapeutic agent(s) and the relative timings of administration will be selected in order to achieve the desired combined therapeutic effect.

### Methods of Screening

The invention further relates to a method of screening for compounds binding to a lipid kinase comprising

(a) binding a compound of formula (I), wherein R¹ is aminoethyl, acryloyl or acryloylaminoethyl carrying a linker and a tag, to a lipid kinase;
(b) mixing with a compound to be screened for binding to said lipid kinase;
(c) measuring displacement of said compound of formula (I) based on the property of the tag; and
(d) calculating binding of the compound to be screened from the result of the measured displacement.

A lipid kinase may be any of the kinases mentioned hereinbefore, in particular phosphoinositide 3-kinase (PI3K), mammalian target of rapamycin (mTOR), DNA-PK and ATM kinase, more specifically PI3K isoform.

Mixing with a compound to be screened according to step (b) can be in many different experimental set-ups. For example the compound to be screened can be added to the complex formed according to step (a) from the lipid kinase and the compound of formula (I) according to the invention by titration, in one batch in excess, or in one batch in less than equimolar amounts, and the displacement determined with a variety of methods depending on the property of the tag, also in a time-dependent manner. If the tag is a fluorophore, the amount of the fluorophore can be measured, or the diffusion of the compound carrying the fluorophore can be measured, which is dependent on the molecular weight of the compound carrying the fluorophore and its interactions with other molecules. It is also possible to use FRET systems with fluorescence quenchers, and other spectroscopic methods well known in the art. If the tag is biotin, any measurable label can be attached through conjugation with avidin, then allowing avidin to bind to biotin. With such an additional step, any such measurable label may be used in the screening method of the invention.

### EXAMPLES

Compounds 5-7, 11 and 12 are synthesized following the procedure in Scheme 2:

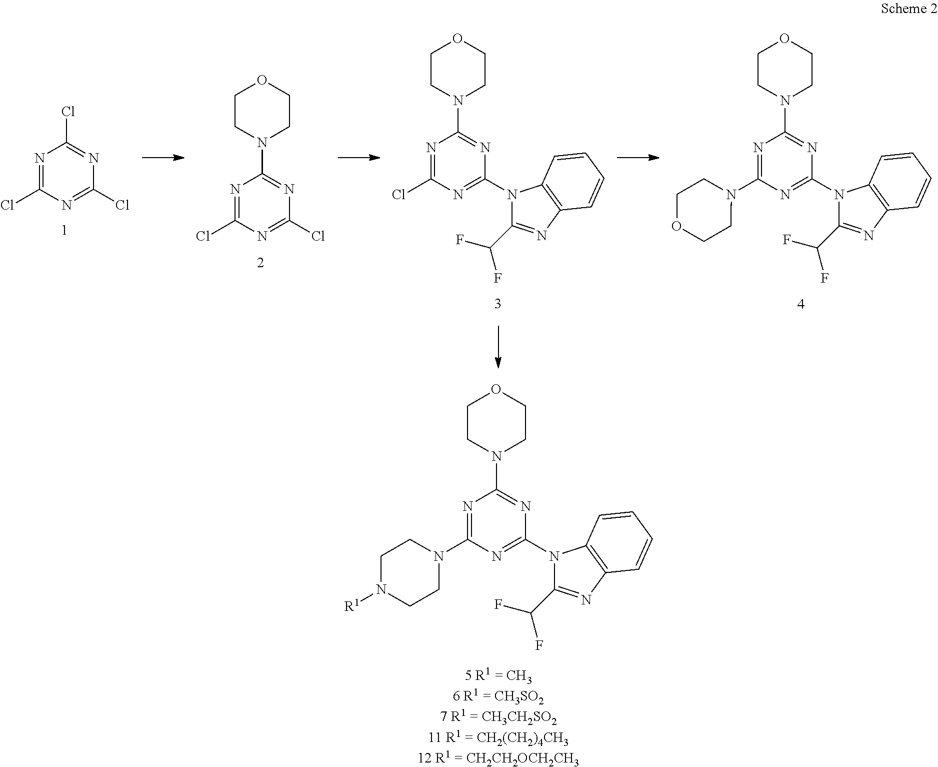

Cyanuric chloride 1 was substituted by morpholine in methylene chloride, at −50° C. for 20 min to give intermediate 2. Replacement of the second chloride with 2-difluoromethyl-1H-benzoimidazole in presence of K₂CO₃ in DMF, at −5° C. for 30 min and further stirring at room temperature for 4 h led to intermediate 3. The final step gave product 4-7, 11 and 12 by amination of intermediate 3 in presence of K₂CO₃ and DMF at room temperature for 45 minutes. Compound 4 is the known compound ZSTK474 prepared for comparison purposes.

Compound 8 is obtained from intermediate 3 by amination with BOC-protected piperazine, followed by BOC deprotection and reaction with acrylic acid anhydride. Compound 9 is obtained from intermediate 3 by amination with 2-(2-(piperazin-1-yl)ethyl)isoindoline-1,3-dione, followed by reaction with hydrazine to split off the phthalimide protecting group. Compound 9 is then treated with acrylic acid anhydride to give compound 10.

The inhibitor efficacy and the cell permeability of the compounds of the invention are measured by in cell Western inhibition assay on melanoma cell line A2058 and TSC2−/− MEFs cell line. Furthermore, in vitro PI3Kalpha inhibition is measured (Table 1). The results are compared with the closest compounds of the state of the art: ZSTK474 (4), a development compound of Zenyaku (compound 4 of EP 1 864 665), and compound 17 lacking the difluoromethyl group (compound 39 of EP 1 020 462).

Using N-methylpiperazine to give compound 5 and sulphonyl containing piperazine to give compounds 6 and 7 had unexpected positive effects on their inhibitor activity against PI3K. Increasing of inhibitor activity against PI3K, but diminishing of selectivity to mTOR was obtained in compound 5. Replacement with more acidic and more soluble 1-(methylsulfonyl)piperazine to give 6 led to excellent inhibitor activity in melanoma cancer cell lines. When 1-(ethanesulfonyl)piperazine was introduced to give compound 7, biological activity against PI3K was slightly decreased comparing to ZSTK474 (4), while selectivity to mTOR became notable reduced.

The activity of compound 5 may also be compared with compound 17, the structurally closest compound of the prior art. Compound 5 is substantially more active than compound 17.

According to molecular modelling experiments, the oxygen atoms of sulfonyl group have the potential to form an additional hydrogen bond with the target protein making this compound more potent. Additionally, due to polarity of the sulfonyl group these compounds have better water solubility than ZSTK474 (4).

Further positive effects were noted when introducing N-n-hexylpiperazine to give compound 11, N-(2-aminoethyl)piperazine and N-(2-ethoxyethyl)piperazine to give compounds 9 and 12, or N-acryloylpiperazine and N-(2-acryloylaminoethyl)piperazine to give compounds 8 and 10, respectively. Introduction of an extended chain carrying a fluorophore connected to N-(2-aminoethyl)piperazine still gave an active compound 13.

TABLE 1
Inhibitor activity^{a}

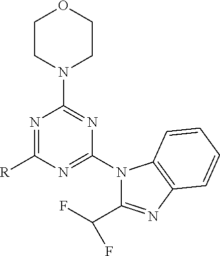

| | | | | | TSC2-/- |
|---|---|---|---|---|---|
| | | | | | MEFs cell |
| | | | A2058 cell inhibition | | inhibition |
| | | In vitro | pPKB/PKB | pS6 | pS6 |
| Compound | R | Pl3Kα | 1 μM | 1 μM | 1 μM |
| 4 ZSTK474 | 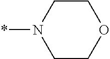 [SMILES: *N1CCOCC1] | 13 | 3.63 ± 1.10 | 22.5 ± 0.13 | 37.9 ± 4.21 |
| 5 | 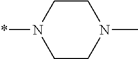 [SMILES: *N1CCN(C)CC1] | 33 | 4.13 ± 0.37 | 18.5 ± 1.70 | 67.3 ± 1.26 |
| 6 | 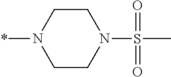 [SMILES: *N1CCN(S(C)(=O)=O)CC1] | 17 | 9.59 ± 2.96 | 19.0 ± 0.54 | 42.3 ± 5.55 |
| 7 | 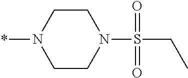 [SMILES: *N1CCN(S(=O)(=O)CC)CC1] | 35 | 30.7 ± 1.08 | 56.7 ± 10.6 | 104 ± 4.27 |
| 8 | 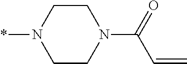 [SMILES: *N1CCN(C(=O)C=C)CC1] | 8 | 13.0 ± 0.01 | 23.0 ± 4.22 | 95.0 ± 7.44 |
| 9 | 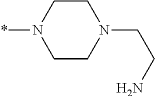 [SMILES: *N1CCN(CCN)CC1] | 50 | 16.5 ± 0.24 | 30.9 ± 4.06 | 89.9 ± 9.50 |
| 10 | 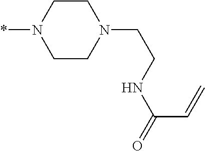 [SMILES: *N1CCN(CCNC(=O)C=C)CC1] | 20 | 3.99 ± 1.18 | 12.7 ± 1.69 | 74.5 ± 5.90 |
| 11 | 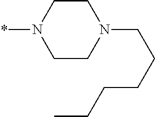 [SMILES: *N1CCN(CCCCCC)CC1] | 45 | 85.2 ± 3.71 | 74.0 ± 10.3 | 85.7 ± 5.71 |
| 12 | 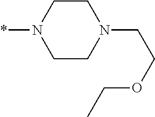 [SMILES: *N1CCN(CCOCC)CC1] | 42 | 15.9 ± 2.46 | 24.4 ± 4.34 | 77.1 ± 4.62 |
| 13 | 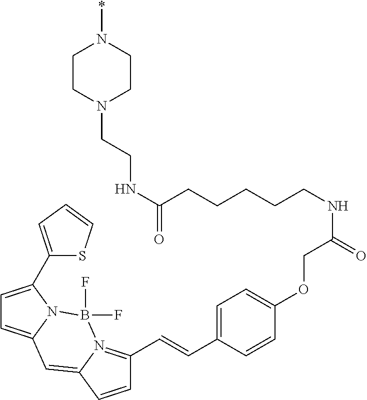 | 13 | 47.5 ± 5.48 | 57.3 ± 6.86 | 95.5 ± 9.81 |
| | 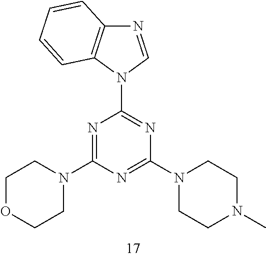 [SMILES: CN1CCN(c2nc(N3CCOCC3)nc(-n3cnc4ccccc43)n2)CC1] | 83 | 122 ± 2.58 | 83.0 ± 3.31 | 123 ± 5.78 |

^{a}Inhibitor efficacy and their cell permeability are measured by in cell Western inhibition assay on melanoma cell line A2058 and TSC2-/-MEFs cell line; in vitro Pl3Kalpha inhibition is measured by Kinase Glo assay at 200 nM; given numbers represent % remaining activity, the smaller the value, the stronger is the inhibition.

### 4-(4,6-Dichloro-1,3,5-triazin-2-yl)morpholine (2)

Cyanuric chloride (10.0 g, 54.2 mmol, 1.0 eq.) is dissolved in methylene chloride (60 ml), and morpholine (4.70 ml, 54.2 mmol, 1.0 eq.) is added drop by drop to the reaction mixture at −50° C., stirred for 20 min at the same temperature and poured into water. After extraction with methylene chloride and ethyl acetate (two times), the organic layers are dried over MgSO₄ and concentrated. Further purification is done by silica gel flash column chromatography (70% hexane/ethyl acetate) to yield the title compound as a colorless solid (3.60 g, 28%). R_{F}: 0.72 (hexane/ethyl acetate, 1:1 v/v); ¹H NMR (CDCl₃, 400 MHz) δ 3.89-3.87 (m, 4H), 3.76-3.74 (m, 4H); ¹³C NMR (CDCl₃, 100 MHz) δ 170.85, 164.50, 66.79, 44.87.

### 4-(4-Chloro-6-(2-(difluoromethyl)-1H-benzo[d]imidazol-1-yl)-1,3,5-triazin-2-yl)morpholine (3)

Compound 2 (425 μmol, 1.0 eq.) is dissolved in DMF (2 ml) and cooled to −5° C., treated with anhydrous potassium carbonate (1.44 eq.) and 2-(difluoromethyl)-1H-benzo[d]-imidazole (1.4 eq.), stirred for 30 min and further stirred at room temperature for 4 h. The reaction mixture is diluted with water and the precipitate is filtered and washed with small amounts of water. Purification is done by silica gel flash column chromatography. R_{F}: 0.72 (methylene chloride/methanol, 95:5 v/v); ¹H NMR (CDCl₃, 400 MHz) δ 8.43 (d, J=7.8 Hz, 1H), 7.90 (d, J=7.6 Hz, 1H), 7.71-7.43 (m, 3H), 4.00-3.95 (m, 4H), 3.86-3.80 (m, 4H); ¹⁹F NMR (CDCl₃, 400 MHz) δ −119.20 (d, J=53.9 Hz, 2 F).

General Procedure for the Reaction of Intermediate 3 with Secondary Amines

Intermediate 3 (270 μmol, 1.0 eq.) is dissolved in DMF (5.4 ml). K₂O₃ (3.2 eq.) and a cyclic secondary amine (1.2 eq.) are added and the reaction mixture is stirred at room temperature for 45 min to 2 h. The reaction mixture is condensed under reduced pressure. The obtained residue is dissolved in methylene chloride and extracted with water. The separated organic layer is washed with water and dried over MgSO₄. The solvent is removed under reduced pressure and the obtained residue is purified via silica gel flash column chromatography.

### 4,4′-(6-(2-(Difluoromethyl)-1H-benzo[d]imidazol-1-yl)-1,3,5-triazine-2,4-diyl)dimorpholine (4), ZSTK474

Following the general procedure, intermediate 3 (100 mg, 270 μmol, 1.0 eq.) is coupled with morpholine (28.0 μl, 320 μmol, 1.2 eq.) for 45 min. Extraction with methylene chloride and water yields the title compound as a colorless solid (110 mg, 96%). R_{F}: 0.45 (methylene chloride/methanol, 95:5 v/v); ¹H NMR (CDCl₃, 400 MHz) δ 8.34-8.32 (m, 1H), 7.90-7.88 (m, 1H), 7.56 (t, J=53.6 Hz, 1H), 7.46-7.37 (m, 2H), 3.88-3.79 (m, 16H); ¹³C NMR (CDCl₃, 100 MHz) δ 165.41, 162.41, 146.60, 142.36, 133.97, 126.19, 124.81, 121.76, 116.23, 108.83, 106.44, 67.04, 44.46, 44.37; ¹⁹F (CDCl₃, 400 MHz) δ −118.41 (d, J=53.9 Hz, 2 F).

### 4-(4-(2-(Difluoromethyl)-1H-benzo[d]imidazol-1-yl)-6-(4-methylpiperazin-1-yl)-1,3,5-triazin-2-yl)morpholine (5)

Following the general procedure, intermediate 3 (100 mg, 270 μmol, 1.0 eq.) is coupled with N-methylpiperazine (36.0 μl, 320 μmol, 1.2 eq.) for 45 min. The obtained residue is purified via silica gel flash column chromatography (5% methanol/methylene chloride) to yield the title compound as a colourless solid (100 mg, 85%). R_{F}: 0.11 (methylene chloride/methanol, 97:3 v/v); ¹H NMR (CDCl₃, 400 MHz) δ 8.33 (d, J=8.1 Hz, 1H), 7.89 (d, J=8.6 Hz, 1H), 7.71-7.37 (m, 3H), 3.93-3.87 (m, 8H), 3.79-3.78 (m, 4H), 2.52 (br. s, 4H), 2.38 (s, 3H); ¹³C NMR (CDCl₃, 100 MHz) δ 165.46, 165.17, 142.36, 126.15, 124.76, 121.73, 116.25, 67.08, 55.12, 46.48; ¹⁹F (CDCl₃, 400 MHz) δ −118.43 (d, J=53.9 Hz, 2 F); ESI-MS (C₂₀H₂₄F₂N₈O): Calc'd. 431.21 (M⁺). Found 431.40.

### 4-(4-(2-(Difluoromethyl)-1H-benzo[d]imidazol-1-yl)-6-(4-(methylsulfonyl)piperazin-1-yl)-1,3,5-triazin-2-yl)morpholine (6)

Following the general procedure, intermediate 3 (70.0 mg, 191 μmol, 1.0 eq.) is coupled with 1-methanesulfonylpiperazine (37.6 mg, 229 μmol, 1.2 eq.) for 45 min. Extraction with methylene chloride yields the title compound as a colourless solid, which is used without further purification (90.0 mg, 95%). R_{F} 0.60 (methylene chloride/methanol 97:3 v/v); ¹H NMR (CDCl₃, 400 MHz) δ 8.29 (d, J=8.2 Hz, 1H), 7.87 (d, J=7.1 Hz, 1H), 7.64-7.36 (m, 3H), 4.01 (br. s, 4H), 3.87-3.78 (m, 8H), 3.33-3.32 (m, 4H), 2.80 (s, 3H); ¹³C NMR (CDCl₃, 100 MHz) δ 165.38, 165.36, 162.44, 146.50, 146.24, 145.97, 142.32, 133.90, 126.30, 124.90, 121.77, 116.17, 111.25, 108.87, 106.48, 67.02, 47.12, 45.94, 44.52, 44.40, 35.15; ¹⁹F (CDCl₃, 400 MHz) δ −117.47 (d, J=53.9 Hz, 2 F); ESI-MS (C₂₀H₂₄F₂N₈O₃S): Calc'd. 495.18 (M⁺). Found 495.10.

### 4-(4-(2-(Difluoromethyl)-1H-benzo[d]imidazol-1-yl)-6-(4-(ethylsulfonyl)piperazin-1-yl)-1,3,5-triazin-2-yl)morpholine (7)

Following the general procedure, intermediate 3 (70.0 mg, 191 μmol, 1.0 eq.) is coupled with 1-(ethanesulfonyl)piperazine (40.8 mg, 229 μmol, 1.2 eq.) for 45 min. Extraction with methylene chloride and water yields the title compound as a colourless solid, which is used without further purification (95.0 mg, 98%). R_{F}: 0.50 (methylene chloride/methanol 97:3 v/v); ¹H NMR (CDCl₃, 400 MHz) δ 8.32-8.29 (m, 1H), 7.90-7.88 (m, 1H), 7.66-7.38 (m, 3H), 3.99 (br. s, 4H), 3.88-3.79 (m, 8H), 3.41-3.39 (m, 4H), 3.02-2.97 (m, 2H), 1.39 (t, J=7.4 Hz, 3H); ¹³C NMR (CDCl₃, 100 MHz) δ 165.41, 142.36, 126.29, 124.91, 121.83, 116.14, 110.48, 67.03, 44.71, 26.92, 8.23; ¹⁹F (CDCl₃, 400 MHz) δ −117.48 (d, J=53.9 Hz, 2 F); ESI-MS (C₂₁H₂₆F₂N₈O₃S): Calc'd. 509.19 (M⁺), 531.18 (M+Na)⁺. Found 509.10, 531.00.

Compound 8 is prepared according to Scheme 3:

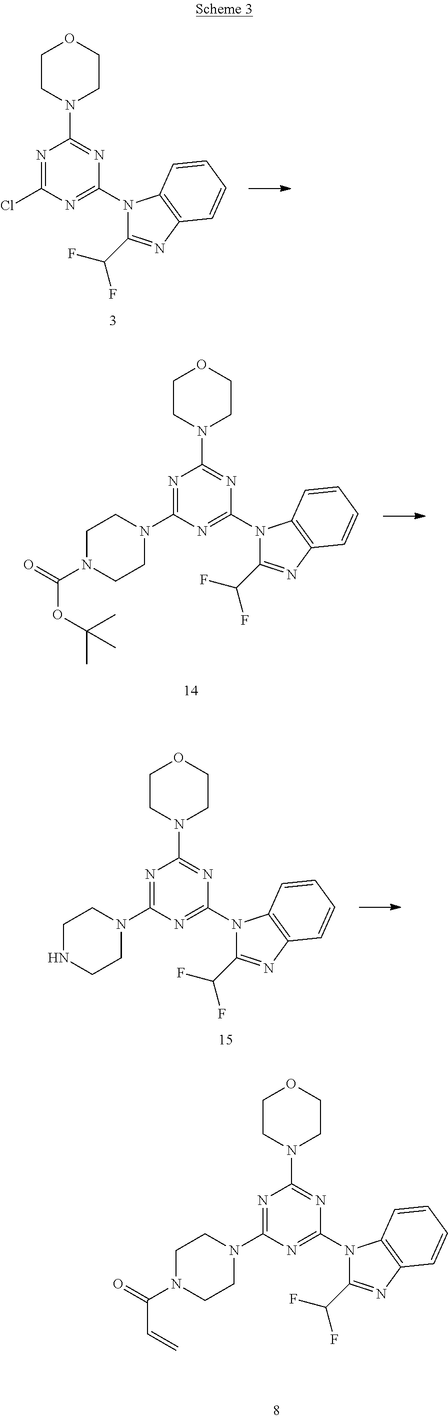 [SMILES: FC(F)c1nc2ccccc2n1-c1nc(Cl)nc(N2CCOCC2)n1 ; CC(C)(C)OC(=O)N1CCN(c2nc(N3CCOCC3)nc(-n3c(C(F)F)nc4ccccc43)n2)CC1 ; FC(F)c1nc2ccccc2n1-c1nc(N2CCNCC2)nc(N2CCOCC2)n1 ; C=CC(=O)N1CCN(c2nc(N3CCOCC3)nc(-n3c(C(F)F)nc4ccccc43)n2)CC1]

### 1-(4-(4-(2-(Difluoromethyl)-1H-benzo[d]imidazol-1-yl)-6-morpholino-1,3,5-triazin-2-yl)piperazin-1-yl)prop-2-en-1-one (8)

To a solution of 4-(4-(2-(difluoromethyl)-1H-benzo[d]imidazol-1-yl)-6-(piperazin-1-yl)-1,3,5-triazin-2-yl)morpholine (15) (100 mg, 240 μmol, 1.0 eq., obtained from intermediate 3 and BOC-protected piperazine according to the general procedure followed by deprotection) in methylene chloride (4.0 ml) are added N,N-diisopropylethylamine (46.0 μl, 264 μmol, 1.1 eq.) and acrylic anhydride (27.7 μl, 240 μmol, 1.0 eq.). The reaction mixture is stirred for 2 h at room temperature. Solvent evaporation and purification via silica gel flash column chromatography (2% methanol/methylene chloride) yields the title compound as a colourless solid (100 mg, 88%). R_{F}: 0.20 (methylene chloride/methanol, 97:3 v/v); ¹H NMR (CDCl₃, 400 MHz) δ 8.30 (d, J=7.58 Hz, 1H), 7.87-7.85 (m, 1H), 7.66-7.35 (m, 3H), 6.63-6.56 (m, 1H), 6.34 (dd, J=1.8, 16.7 Hz, 1H), 5.75 (dd, J=1.8, 10.6 Hz, 1H), 3.90-3.86 (m, 8H), 3.79-3.67 (m, 8H); ¹⁹F (CDCl₃, 400 MHz) δ; ESI-MS (C₂₂H₂₄F₂N₈O₂): Calc'd. 493.20 (M+Na)⁺. Found 493.20.

Compound 9 and 10 are prepared according to Scheme 4.

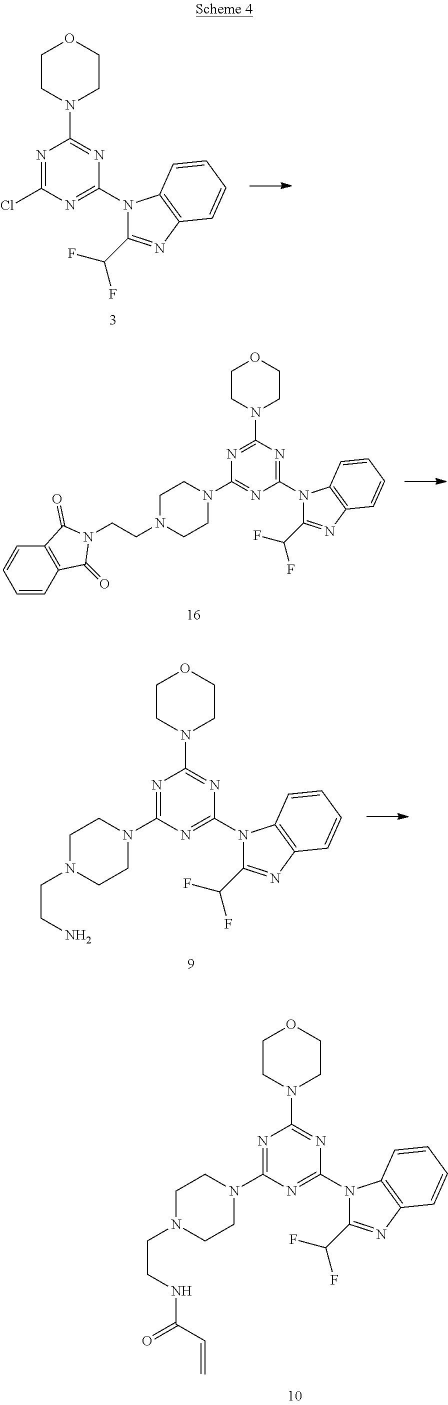 [SMILES: FC(F)c1nc2ccccc2n1-c1nc(Cl)nc(N2CCOCC2)n1 ; O=C1c2ccccc2C(=O)N1CCN1CCN(c2nc(N3CCOCC3)nc(-n3c(C(F)F)nc4ccccc43)n2)CC1 ; NCCN1CCN(c2nc(N3CCOCC3)nc(-n3c(C(F)F)nc4ccccc43)n2)CC1 ; C=CC(=O)NCCN1CCN(c2nc(N3CCOCC3)nc(-n3c(C(F)F)nc4ccccc43)n2)CC1]

### 2-(4-(4-(2-(Difluoromethyl)-1H-benzo[d]imidazol-1-yl)-6-morpholino-1,3,5-triazin-2-yl)piperazin-1-yl)ethanamine (9)

2-(2-(4-(4-(2-(Difluoromethyl)-1H-benzo[d]imidazol-1-yl)-6-morpholino-1,3,5-triazin-2-yl)piperazin-1-yl)ethyl)isoindoline-1,3-dione (16) (70 mg, 119 μmol, 1.0 eq., obtained from intermediate 3 and 2-(2-(piperazin-1-yl)ethyl)isoindoline-1,3-dione) is taken up in ethanol (326 μl) and treated with hydrazine monohydrate (6.4 μl, 130 μmol, 1.1 eq.). The resulting mixture is refluxed at 100° C. for 5 h, whereupon a white precipitate formed. The slurry is allowed to cool and then treated with conc. hydrochloric acid (28.4 μl) followed by refluxing again for 1 h. The slurry is allowed to cool to room temperature and the white solid is filtered off. The filtrate is evaporated in vacuo and the residue taken up in water (1.5 ml) and the solution brought to pH 11 with 1 M NaOH. The aqueous phase is saturated with NaCl and extracted with methylene chloride. The combined organic phases are dried over MgSO₄, evaporated, and dried in vacuo to obtain the title compound as a colourless solid. R_{F}: 0.00 (ethyl acetate/methylene chloride, 1:1 v/v); ¹H NMR (CDCl₃, 400 MHz) δ 8.33 (d, J=7.3 Hz, 1H), 7.87 (d, J=7.6 Hz, 1H), 7.57 (t, J=53.6 Hz, 1H), 7.44-7.36 (m, 2H), 3.88-3.85 (m, 8H), 3.78-3.77 (m, 4H), 2.84 (t, J=6.1 Hz, 2H), 2.53-2.47 (m, 6H); ¹³C NMR (CDCl₃, 100 MHz) δ 165.45, 165.08, 162.38, 146.36, 142.34, 133.98, 126.12, 124.73, 121.69, 116.27, 108.82, 72.98, 70.91, 70.32, 67.11, 67.05, 62.17, 53.37, 39.07; ¹⁹F (CDCl₃, 400 MHz) δ −118.44 (d, J=53.9 Hz, 2 F); ESI-MS (C₂₁H₂₇F₂N₉O): Calc'd. 460.24 (M⁺). Found 460.20.

### N-(2-(4-(4-(2-(Difluoromethyl)-1H-benzo[d]imidazol-1-yl)-6-morpholino-1,3,5-triazin-2-yl)piperazin-1-yl)ethyl)acrylamide (10)

To a solution of compound 9 (100 mg, 218 μmol, 1.0 eq.) in methylene chloride (4.0 ml) are added N,N-diisopropylethylamine (46.0 μl, 264 μmol, 1.1 eq.) and acrylic anhydride (38.0 μl, 218 μmol, 1.0 eq.). The reaction mixture is stirred for 2 h at room temperature. Solvent evaporation and further purification via preparative thin layer chromatography (10% methanol/ethyl acetate) yields the title compound as colourless solid (53 mg, 47%). R_{F}: 0.40 (ethyl acetate/methanol, 9:1 v/v); ¹H NMR (CDCl₃, 500 MHz) δ 8.32 (d, J=8.2 Hz, 1H), 7.87 (d, J=7.9 Hz, 1H), 7.66-7.36 (m, 3H), 6.31-6.28 (m, 2H), 6.17-6.11 (m, 1H), 5.65 (d, J=10.4 Hz, 1H), 3.89-3.86 (m, 8H), 3.77 (br. s, 4H), 3.52-3.49 (m, 2H), 2.62-2.57 (m, 6H); ¹³C NMR (CDCl₃, 125 MHz) δ 165.62, 165.05, 164.77, 162.02, 146.17, 145.96, 145.75, 141.94, 133.58, 130.80, 128.20, 126.54, 125.81, 124.42, 121.31, 115.88, 110.36, 108.45, 106.54, 66.66, 56.65, 52.68, 52.52, 44.07, 43.95, 43.47, 43.26, 35.90; ¹⁹F (CDCl₃, 400 MHz) δ −117.49 (d, J=53.9 Hz, 2 F); ESI-MS (C₂₄H₂₉F₂N₉O₂): Calc'd. 514.25 (M⁺). Found 514.30 and 536.24 (M+Na)⁺. Found 536.20.

### 4-(4-(2-(Difluoromethyl)-1H-benzo[d]imidazol-1-yl)-6-(4-hexylpiperazin-1-yl)-1,3,5-triazin-2-yl)morpholine (11)

Following the general procedure, intermediate 3 (70 mg, 191 μmol, 1.0 eq.) is coupled with 1-hexylpiperazine (44.5 μl, 229 μmol, 1.2 eq.) for 45 min. Extraction with methylene chloride and water yields the title compound as a colourless solid (90.0 mg, 94%). R_{F}: 0.44 (methylene chloride/methanol, 95:5 v/v); ¹H NMR (CDCl₃, 400 MHz) δ 8.35-8.33 (m, 1H), 7.90-7.88 (m, 1H), 7.58 (t, J=53.5 Hz, 1H), 7.45-7.37 (m, 2H), 3.89-3.87 (m, 8H), 3.79-3.77 (m, 4H), 2.51 (s, 4H), 2.38 (t, J=7.7 Hz, 2H), 1.54-1.49 (m, 2H), 1.34-1.28 (m, 6H), 0.89 (t, J=6.8 Hz, 3H); ¹³C NMR (CDCl₃, 100 MHz) δ 165.48, 165.06, 162.39, 126.11, 124.72, 121.71, 116.28, 67.07, 59.19, 53.38, 44.89, 32.19, 27.63, 27.24, 23.02, 14.47; ¹⁹F (CDCl₃, 400 MHz) δ −118.44 (d, J=53.9 Hz, 2 F); ESI-MS (C₂₅H₃₄F₂N₈O): Calc'd. 501.29 (M⁺). Found 501.30.

### 4-(4-(2-(Difluoromethyl)-1H-benzo[d]imidazol-1-yl)-6-(4-(2-ethoxyethyl)piperazin-1-yl)-1,3,5-triazin-2-yl)morpholine (12)

Following the general procedure, intermediate 3 (70 mg, 191 μmol, 1.0 eq.) is coupled with 1-(2-ethoxyethyl)piperazine (38.5 μl, 229 μmol, 1.2 eq.) for 45 min. Extraction with methylene chloride and water yields the title compound as a colourless oil (90.0 mg, 96%). R_{F}: 0.28 (methylene chloride/methanol, 95:5 v/v); ¹H NMR (CDCl₃, 400 MHz) δ 8.35-8.33 (m, 1H), 7.89-7.87 (m, 1H), 7.58 (t, J=53.7 Hz, 1H), 7.45-7.37 (m, 2H), 3.90-3.86 (m, 8H), 3.79-3.77 (m, 4H), 3.60 (t, J=5.7 Hz, 2H), 3.55-3.50 (m, 2H), 2.65 (t, J=5.7 Hz, 2H), 2.60-2.59 (m, 4H), 1.22 (t, J=7.1 Hz, 3H); ¹³C NMR (CDCl₃, 100 MHz) δ 165.46, 165.06, 162.39, 142.36, 134.00, 126.11, 124.73, 121.70, 116.27, 108.82, 68.47, 66.96, 58.37, 53.71, 15.57; ¹⁹F (CDCl₃, 400 MHz) δ −118.43 (d, J=53.9 Hz, 2 F); ESI-MS (C₂₃H₃₀F₂N₈O₂): Calc'd. 489.26 (M⁺). Found 489.20.

### (E)-3-(4-(2-((6-((2-(4-(4-(2-(Difluoromethyl)-1H-benzo[d]imidazol-1-yl)-6-morpholino-1,3,5-triazin-2-yl)piperazin-1-yl)ethyl)amino)-6-oxohexyl)amino)-2-oxoethoxy)styryl)-5,5-difluoro-7-(thiophen-2-yl)-5H-dipyrrolo[1,2-c:2′,1′-f][1,3,2]diazaborinin-4-ium-5-uide (13)

To a solution of compound 9 (1.0 eq., 6.88 μmol, 3.16 mg) in abs. DMSO-d₆ (100 μl) is added at room temperature Bodipy 630/650-X (Invitrogen, D10000) (5.00 mg, 7.57 μmol, 1.1 eq.) dissolved in 200 μl DMSO-d₆. The reaction mixture is left in the NMR-tube and controlled by NMR. ESI-MS (C₅₀H₅₃BF₄N₁₂O₄S): Calc'd. 1005 (M⁺). Found 1005.

### Use of Fluorophore Containing Compound 13 as Reference Inhibitor for PI3Kgamma Isoform

Fluorophore containing compound 13 can be used as a reference inhibitor by fluorescence correlation spectroscopy experiments in which the binding constants of novel inhibitors are determined. Compound 13 binds to the PI3Kgamma isoform, which leads to an increase in diffusion time. When the enzyme is saturated with compound 13, new possible ligands can be tested by observing the release of compound 13. This leads to a decrease in diffusion time.

1. A compound of formula (I) 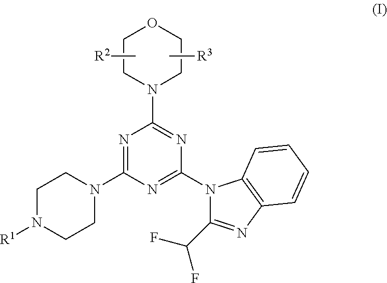
wherein
R¹ is methyl, n-hexyl, aminoethyl, methylaminoethyl, ethylaminoethyl, dimethylaminoethyl, acryloylaminoethyl, methacryloylaminoethyl, methoxyethyl, ethoxyethyl, C₁-C₄-alkylsulfonyl, acryloyl, or methacryloyl; or R¹ is aminoethyl, acryloyl or acryloylaminoethyl carrying a linker and a tag, and
R² and R³, independently of each other, are hydrogen or C₁-C₄-alkyl, or R² and R³ together form an ethylene bridge;
and tautomers, solvates and pharmaceutically acceptable salts thereof.

2. The compound of formula (I) according to claim 1 wherein
R¹ is methyl, n-hexyl, 2-aminoethyl, 2-(methylamino)ethyl, 2-(ethylamino)ethyl, 2-(acryloylamino)ethyl, 2-methoxyethyl, 2-ethoxyethyl, C₁-C₄-alkylsulfonyl, acryloyl, or methacryloyl.

3. The compound of formula (I) according to claim 1 wherein
R¹ is 2-aminoethyl, acryloyl or 2-(acryloylamino)ethyl, each carrying a linker and a tag.

4. The compound of formula (I) according to claim 1 wherein
R¹ is methyl, 2-aminoethyl, 2-(acryloylamino)ethyl, 2-ethoxyethyl, methylsulfonyl, ethylsulfonyl, iso-propylsulfonyl, acryloyl, or methacryloyl.

5. The compound of formula (I) according to claim 1 wherein
R¹ is methyl, 2-aminoethyl, 2-(acryloylamino)ethyl, 2-ethoxyethyl, methylsulfonyl, ethylsulfonyl, or acryloyl.

6. The compound of formula (I) according to claim 1 wherein
R¹ is methyl, 2-(acryloylamino)ethyl, methylsulfonyl, or acryloyl.

7. The compound of formula (I) according to claim 1 wherein
R¹ is 2-aminoethyl or acryloyl, each carrying a linker and a tag selected from biotin, a fluorophore or a polymeric bead.

8. The compound of formula (I) according to claim 1 wherein
R² and R³, independently of each other, are hydrogen, methyl, ethyl, or isopropyl, or R² and R³ together form a methylene or an ethylene bridge.

9. The compound of formula (I) according to claim 1 wherein
R² and R³ are both hydrogen, one hydrogen and one methyl, or both methyl, or form together an ethylene bridge.

10. The compound of formula (I) according to claim 1 wherein
R² is (S)-2-methyl, (R)-2-methyl, (R)-3-methyl, or (S)-3-methyl, and R³ is hydrogen, or R² and R³ are (2R,6S)-2,6-dimethyl, (2R,6R)-2,6-dimethyl, (2R,3R)-2,3-dimethyl, (2S,5S)-2,5-dimethyl, (3S,5R)-3,5-dimethyl, (3S,5S)-3,5-dimethyl, a 2,5-ethylene bridge, a 2,6-ethylene bridge, a 3,5-ethylene bridge, or both hydrogen.

11. The compound of formula (I) according to claim 1 wherein R¹ is methyl, n-hexyl, 2-aminoethyl, 2-(acryloylamino)ethyl, 2-ethoxyethyl, methylsulfonyl, ethylsulfonyl, or acryloyl, and R² and R³ are hydrogen.

12. The compound of formula (I) according to claim 1 wherein R¹ is methyl, 2-(acryloylamino)ethyl, methylsulfonyl, or acryloyl, and R² and R³ are hydrogen.

13. The compound of formula (I) according to claim 1 wherein R¹ is methyl or methylsulfonyl, and R² and R³ are hydrogen.

14. A pharmaceutical composition comprising a compound of formula (I) according to claim 1.

15. A method of preventing or treating a disease or disorder modulated by PI3 kinases and/or mTOR comprising administering to a mammal in need of such treatment an effective amount of a compound of formula (I) according to claim 1.

16. A method of treating a hyperproliferative disorder according to claim 15 comprising administering to a mammal in need of such treatment an effective amount of a compound of formula (I).

17-18. (canceled)

19. A method of screening for compounds binding to a lipid kinase comprising
(a) binding a compound of formula (I), wherein R¹ is aminoethyl, acryloyl or acryloylaminoethyl carrying a linker and a tag, to a lipid kinase;
(b) mixing with a compound to be screened for binding to said lipid kinase;
(c) measuring displacement of said compound of formula (I) based on the property of the tag; and
(d) calculating binding of the compound to be screened from the result of the measured displacement.
